(19) United States
(12) Patent Application Publication
(10) Pub. No.: US 2026/0275257 A1
(43) Pub. Date: Sep. 17, 2026

(54) DEVICE AND METHOD FOR ORGANOID CULTURE

(71) Applicant: LUDWIG-MAXIMILIANS-UNIVERSITÄT MÜNCHEN, München (DE)

(72) Inventors: Stefan MUNKER, München (DE); Stefan ZAHLER, Maisach (DE); Miguel SILVA, München (DE)

(21) Appl. No.: 19/472,236

(22) PCT Filed: Feb. 6, 2024
(86) PCT No.: PCT/EP2024/052867

§ 371 Date: Oct. 3, 2025

(30) Foreign Application Priority Data
Apr. 4, 2023 (EP) 23166617.3

(51) Int. Cl. C12M 1/32; C12M 1/00; C12M 1/12; C12M 1/26

(52) U.S. Cl. CPC C12M 23/12; C12M 23/20; C12M 23/22; C12M 23/34; C12M 25/14; C12M 29/20; C12M 33/04

(57) ABSTRACT

The present invention relates to a multi-use specimen carrier for the long term study of cells and tissues in vitro or for co-culture experiments with the usage of extra-cellular matrix embedding and more particularly comprises a new and improved multi-well cell culture plate and a changed wall structure that allows a strong reduction of meniscus formation and increased lateral surface adhesion and stabilization of extracellular matrices, a deep well in a well in well and surrounding well system and additional measures for increased local humidity hereby allowing longer extracellular matrix gel stability and repeated replenishment or change of matrix (sealing) with greatly increased matrix stability and thereby allowing long term culture and coculture of cells and tissues.

### FIELD OF THE INVENTION

The present invention relates to a multi-use specimen carrier for the long term study of cells and tissues in vitro or for co-culture experiments with the usage of extracellular matrix embedding and more particularly comprises a new and improved multiwell cell culture plate and a changed wall structure that allows a strong reduction of meniscus formation and increased lateral surface adhesion and stabilization of extracellular matrices, a deep well in a well in well and surrounding well system and additional measures for increased local humidity hereby allowing longer extracellular matrix gel stability and repeated replenishment or change of matrix (sealing) with greatly increased matrix stability and thereby allowing long term culture and coculture of cells and tissues.

### BACKGROUND OF THE INVENTION

Advantages of growing tissue or cells in vitro embedded in extracellular matrix have been known. Usage of extracellular matrix such as the BD Matrigel from Boston or iterations of extracellular matrix related matrices resemble the basement membrane composition of epithelial cells or extracellular matrix of mesenchymal cells in vivo. The type of carrier material is generally also known as matrix. These matrices allow cells or tissues to form cells sheets resembling circular structures the so-called organoids or tumoroids. These in vitro cells and disease avatars are considered highly relevant for preclinical research capable of replacing in vivo models of many diseases to a certain extent. Systems that allow long term and stable gel formation are advantageous in tumoroid and organoid culture.

Culturing cells in between layers of extracellular matrix with embedding of cells or tissues in between is called the Sandwich embedding method, which more closely resembles in vivo conditions by retaining cellular polarity. Putting additional matrix layers of different matrices is currently not possible without the prevention of meniscus formation and increased matrix stability.

Exposition of matrix to strong liquid currents during media changes can damage the matrix. Also, the matrix is often degraded enzymatically during long term cell culture experiments on these cells. It is then necessary to remove the cells from the matrix and resuspend them in new matrix, which also poses the risk of losing cells or exerting detrimental stress on the cells.

Microscopic read-outs for cell culture studies are also possible with matrix embedded cells. The problem of microscopy of matrix embedded cells is that the matrix distributes itself uncontrollably and undesirable meniscus formation occurs. Uneven Matrix geometry and Meniscus formation of the gels at the well-wall or well-corners interface limits the usage of matrix in smaller well-plate formats, such as 48- 96- and 384 well plates. In the boundary area of the wells the meniscus increases the refractive index limiting microscopic read-outs. Well in well systems have been used to allow gel formation of defined liquid volume with controllable meniscus formation, for example U.S. Pat. No. 8,263,391B2. These systems however do not allow seamless changes in volumes with decreased meniscus formation with partial filling of the wells or adding further matrix layers on top without meniscus formation.

Local media evaporation increases osmolarity of the media over cell culture time making more frequent media changes necessary and potentially have detrimental effects on cell culture conditions.

There is hence a need for an improved device suitable for gel stabilization and organoid culture, as well as corresponding methods.

### OBJECTS AND SUMMARY OF THE INVENTION

The present invention addresses this need and provides, in one aspect, a specimen carrier, comprising one or more reservoirs, wherein said reservoirs comprise a segmented inner wall structure (meniscus barrier) which laterally stabilizes an enclosed gel and prevents the formation of a meniscus of a liquid or gel matrix material filled into the reservoir. This specimen carrier advantageously allows to significantly increase gel stability. The present invention thus provides a device comprising segmented walls allowing near seamless matrix filling and greatly decreasing the probability of generation of a meniscus, wherein simultaneously the matrix stability is increased. By providing repeated segmentations, also the lateral contact surface area of the filled in gel is increased, which elevates the adhesion force of the gel and thereby introduces an improved lateral mechanical stabilization. Further mechanical protection from the top of the gel matrix is achieved by the possibility of adding new layers of matrix (also other matrices that may be stiffer) on top of the matrix without meniscus formation. Further physical protection from currents can be achieved by a deep well in well structure.

The present invention thus relates to a multi-use system that allows layering of matrix deposition without meniscus formation in a reservoir for long-term cell culture and co-culture studies of tumoroid, organoids or stem cells. It further allows media change with decreased mechanical strain on the embedded matrix and it increases local humidity due to a separate water reservoir. These measures altogether increase matrix stability and cell viability.

In a preferred embodiment the inner wall structure comprises at least two, preferably a multitude of, preferably 10 to 20, vertically arranged meniscus barrier(s), wherein said barriers are designed to prevent near seamless formation of a meniscus of liquids or gel matrix material filled into said reservoir and to stabilize the gel by increasing the contact surface and to strengthen lateral adhesion.

In a further embodiment said meniscus barrier is vertical, circular, linear, mechanical and/or a chemical meniscus barrier, or a combination thereof, preferably with a vertical distance of 40 μm-2 mm to further barriers.

In a further embodiment said mechanical meniscus barrier is an inner wall indentation, an inner wall protuberance, preferably a pointed or curved protuberance, a recess or shoulder, preferably a shoulder comprising an angle of about 90° or less than 90°, or more than 90°.

Said mechanical meniscus barriers may comprise a sequence of orthogonal steps. Advantageously, shoulders/steps can easily be produced with injection moulding. Therefore, this embodiment is preferred.

In yet another preferred embodiment said shoulder meniscus barrier has a horizontal dimension of 5 μm to 2 mm, preferably 5 μm to 500 μm, and a vertical dimension 5 μm to 2 mm.

It is further preferred that the shoulder meniscus barriers comprise a vertical tip, preferably of an angle of 1-15° and/or a base width of 1 μm-100 μm.

In yet another preferred embodiment said chemical barrier is an alternating hydrophobic and hydrophilic surface of the wall.

In another preferred embodiment a mechanical and a chemical barrier is combined.

In yet another preferred embodiment of the specimen carrier the inner wall of the reservoir comprises a coating, preferably a coating which is present at specific zones of the inner wall, more preferably at repeating zones followed by non-coated zones in vertical direction.

In yet another preferred embodiment the reservoir comprises an inner reservoir (11) and an outer reservoir (12), wherein the inner reservoir comprises the inner wall structure which prevents the formation of meniscus formation of liquids or gel matrix.

It is particularly preferred that the inner reservoir (11) is designed to receive a gel matrix and optionally cell culture medium (matrix reservoir).

It is particularly preferred that the outer reservoir (12) is designed to receive cell culture medium (medium reservoir).

In a further preferred embodiment the cell culture medium present in the outer reservoir overlays a matrix layer present in the inner reservoir.

In yet another preferred embodiment the reservoir, more preferably the medium reservoir (12) comprises a ventilation opening (1).

It is further preferred that the inner reservoir (11) and the outer reservoir (12) are in liquid or aerial communication with an additional liquid reservoir, preferably an adjacent liquid reservoir.

It is further preferred that the medium reservoir (12) is in liquid communication with the matrix reservoir (11).

In a further preferred embodiment the carrier comprises a liquid distribution system adjacent to the reservoir(s) which is in liquid or aerial communication with all reservoirs of the carrier, preferably through ventilation openings (1).

In another preferred embodiment the carrier is a multi-well carrier, preferably a 12, 24, 48 or 96 well carrier.

The present invention further envisages that the reservoir has a circular, rectangular, trapezoid, ellipsoid, or roundly shaped transversal form.

According to a preferred embodiment the reservoir of a circular transversal form has a cylindric or conical shape, or wherein the rectangular transversal form has a cuboidal or pyramidal shape.

It is further preferred that the carrier comprises an access port for liquid injection, preferably at an edge or corner of the carrier.

It is additionally preferred that the inner reservoir has a symmetric or asymmetric position with respect to the outer reservoir in plan view, preferably having a circular, rectangular, trapezoid, ellipsoid, or roundly shaped form in plan view.

In a preferred embodiment said asymmetric position is an eccentric position with respect to the center of the outer reservoir in plan view.

In a further preferred embodiment the bottom of the reservoir, preferably of the inner (11) and/or outer reservoir (12), is transparent, preferably designed for microscopy analysis.

In a further aspect the present invention relates to the use of the specimen carrier as defined above for culturing cells.

According to preferred embodiments the specimen carrier is used under suitable culture conditions, such as 37° C. and 100% humidity.

In a further aspect the present invention relates to a method for the culturing of cells, comprising: (i) providing a specimen carrier as defined above; (ii) filling the reservoirs with matrix gel and optionally culture liquid; (iii) culturing cells within the specimen carrier, preferably for a time period of 1, 2, 3 days up to 4, 8, or 12 weeks.

Further envisaged is a method for the microscopic analysis of living cells or tissues under culture conditions, comprising: (i) culturing cells within the specimen carrier providing a specimen carrier as defined above; (ii) performing microscopic analysis of cells embedded in a gel matrix in the specimen carrier.

### BRIEF DESCRIPTION OF THE DRAWINGS

FIG. 1 provides a schematical illustration of an embodiment of the present invention. In FIG. 1A a specimen carrier with one to several adjacent reservoirs is shown. Further the direction of a cross-sectional cut in one reservoir is indicated. FIG. 1B shows one of the reservoirs in a longitudinal cut. The specimen carrier comprises a liquid reservoir (5), e.g., for distilled water, a cell culture medium (2), optionally a ventilation opening (1) which allows circulation of humidity saturated air, a matrix reservoir (11), and an adjacent medium reservoir (12). Accordingly, the space adjacent to the matrix reservoir can be used to increase the medium volume in the well and improve containment of media and feeder cells. Matrix can be layered on the bottom of the reservoir. The growth zone usually contains gel matrix (4) here the cells are cultured and grown. Additional matrix layers (3) may be added. The height of the walls enclosing the medium reservoir is preferably lower than the height of the well or matrix reservoir. This allows for a media exchange if the media crosses the wall height. The matrix reservoir (11) wall may have a height of 100 μm-20 mm

FIG. 2 shows a further embodiment of the present invention, e.g., in the form of a plate well. The matrix reservoir (11) as depicted in FIG. 1B, comprises vertical barriers along the wall of the reservoir in the form of indentations (8). Various volumina of matrix can be pipetted without the formation of a matrix meniscus. The reservoir comprises a growth zone usually containing the gel matrix (4) optionally with several additional layers of matrix, and a liquid medium (2) overlaying said growth zone.

FIG. 3 shows a further embodiment of the present invention, e.g., in the form of a plate well. The matrix reservoir (11) as depicted in FIG. 1B, comprises vertical barriers along the wall of the reservoir in the form of pointed protuberances (9). Various volumina of matrix can be pipetted without the formation of a matrix meniscus. The reservoir comprises a growth zone (4), optionally with several layers of matrix, and a liquid medium (2) overlaying said growth zone.

FIG. 4 shows a further embodiment of the present invention, e.g., in the form of a plate well. The matrix reservoir (11) as depicted in FIG. 1B, comprises vertical barriers along the wall of the reservoir in the form of recesses (13). Various volumina of matrix can be pipetted without the formation of a matrix meniscus. The reservoir comprises a growth zone (4), optionally with several layers of matrix, and a liquid medium (2) overlaying said growth zone. This plurality of recesses or orthogonal steps is suitable for matrix meniscus prevention and increased adhesion and mechanical gel stabilisation. It may have different dimensions (13, 10).

FIG. 5 shows a further embodiment of the present invention, e.g., in the form of a plate well. The matrix reservoir (11) as depicted in FIG. 1B, comprises vertical barriers along the wall of the reservoir in the form of curved protuberances (7). Various volumina of matrix can be pipetted without the formation of a matrix meniscus. The reservoir comprises a growth zone usually containing a gel metrix (4), optionally with several layers of matrix, and a liquid medium (2) overlaying said growth zone.

FIG. 6 shows a further embodiment of reservoir shown in FIG. 4. The matrix reservoir (11) as depicted in FIG. 1B, comprises vertical barriers along the wall of the reservoir in the form of recesses (14), which have a small vertical sharp (e.g., 1-15°) tip, lip or rim (e.g., of a width of 1-100 μm width at the base) on the recesses, which further improves prevention of meniscus formation. Various volumina of matrix can be pipetted without the formation of a matrix meniscus and increased mechanical stability.

FIG. 7 shows a further embodiment according to the present invention. The Fig. depicts an alternative to the reservoir shown in FIG. 1. The reservoir of FIG. 7 does not comprise a medium reservoir zone adjacent to the matrix reservoir.

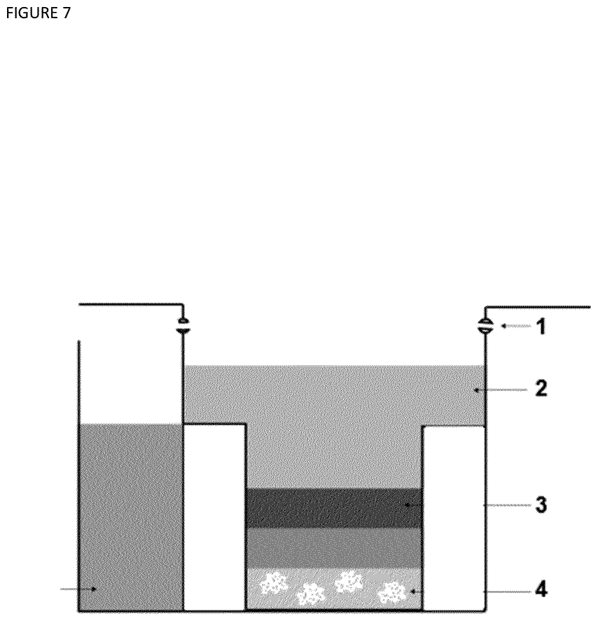

FIG. 8 shows a further embodiment according to the present invention. The reservoirs are shown to be integrated into a multi-well specimen carrier, e.g., in a 96-well format. Also envisaged are other formats such as a 48-well format or any format with higher or lower well numbers. Further shown is the liquid reservoir (5) as already depicted in FIG. 1B. The multi-well specimen carrier may additionally comprise an access port (17) that allows water injection, being located on top of carrier.

FIG. 9 depicts a further alternative embodiment according to the present invention. The matrix reservoir and the adjacent reservoir can different geometric forms such as a circular form (15), to facilitate the access by a pipetting system. Alternatively, the matrix reservoir may be put into an eccentric position (16). The multi-well specimen carrier may additionally comprise an access port (17) that allows water injection, being located on top of carrier to access the humidity reservoir.

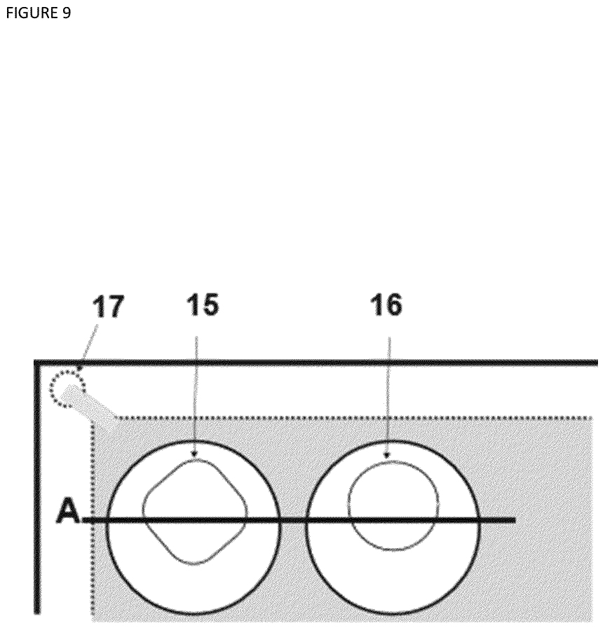

FIG. 10 shows a further embodiment of the present invention, e.g., in the form of a plate well with several pronounced (left hand side) or less pronounced (right hind side) shoulders/steps (18). Various volumina of matrix can be pipetted without the formation of a matrix meniscus and increased lateral adhesion and gel stability. The reservoir comprises a growth zone consisting of a gel matrix, optionally with several layers of matrix on top, and a liquid medium overlaying said gel matrix or bottom. This plurality of shoulders or orthogonal steps is suitable for near seamless matrix meniscus prevention and gel stabilization. It may have different dimensions, e.g., a width of 5-500 μm.

FIG. 11 shows a further embodiment of the present invention, e.g., in the form of a plate well with several shoulders/steps. The matrix reservoir (11) as depicted in FIG. 1B, comprises vertical barriers along the wall of the reservoir in the form of steps/shoulders (13). Various volumina of matrix can be pipetted without the formation of a matrix meniscus. The reservoir comprises a section with the gel matrix (4), optionally with several layers of matrix on top, and a liquid medium (2) overlaying said growth zone. This plurality of shoulders or orthogonal steps (19) is suitable for matrix meniscus prevention. It may have different dimensions (10), e.g., a width of 5-500 μm. In a specific embodiment the rim as depicted in FIG. 6 may also be applied on the shoulders (14).

FIG. 12 shows an illustration of the geometry of meniscus formation wherein alpha is the meniscus arc angle, beta the angle of the said mechanical structure in relation to the surface of the reservoir wall and theta the sharpest angle of the meniscus.

### DETAILED DESCRIPTION OF THE EMBODIMENTS

Although the present invention will be described with respect to particular embodiments, this description is not to be construed in a limiting sense.

Before describing in detail exemplary embodiments of the present invention, definitions important for understanding the present invention are given.

As used in this specification and in the appended claims, the singular forms of “a” and “an” also include the respective plurals unless the context clearly dictates otherwise

In the context of the present invention, the terms “about” and “approximately” denote an interval of accuracy that a person skilled in the art will understand to still ensure the technical effect of the feature in question. The term typically indicates a deviation from the indicated numerical value of ±20%, preferably ±15%, more preferably ±10%, and even more preferably ±5%.

It is to be understood that the term “comprising” is not limiting. For the purposes of the present invention the term “consisting of” or “essentially consisting of” is considered to be a preferred embodiment of the term “comprising of”. If hereinafter a group is defined to comprise at least a certain number of embodiments, this is meant to also encompass a group which preferably consists of these embodiments only.

Furthermore, the terms “(i)”, “(ii)”, “(iii)” or “(a)”, “(b)”, “(c)”, or “first”, “second”, “third” etc. and the like in the description or in the claims, are used for distinguishing between similar elements and not necessarily for describing a sequential or chronological order. It is to be understood that the terms so used are interchangeable under appropriate circumstances and that the embodiments of the invention described herein are capable of operation in other sequences than described or illustrated herein. In case the terms relate to steps of a method or use there is no time or time interval coherence between the steps, i.e., the steps may be carried out simultaneously or there may be time intervals of seconds, minutes, hours, days, weeks etc. between such steps, unless otherwise indicated.

It is to be understood that this invention is not limited to the particular methodology, protocols, reagents etc. described herein as these may vary. It is also to be understood that the terminology used herein is for the purpose of describing particular embodiments only and is not intended to limit the scope of the present invention that will be limited only by the appended claims. Unless defined otherwise, all technical and scientific terms used herein have the same meanings as commonly understood by one of ordinary skill in the art.

The drawings are to be regarded as being schematic representations and elements illustrated in the drawings are not necessarily shown to scale. Rather, the various elements are represented such that their function and general purpose become apparent to a person skilled in the art.

Unless defined otherwise, all technical and scientific terms used herein have the same meanings as commonly understood by one of ordinary skill in the art.

Independent of the grammatical term usage, individuals with male, female or other gender identities are included within the term.

As has been set out above, the present invention concerns in one aspect a specimen carrier, comprising one or more reservoirs, wherein said reservoirs comprise a segmented inner wall structure (meniscus barrier) which laterally stabilizes an enclosed gel that prevents meniscus formation of a liquid or gel matrix material filled into said reservoir. The term “reservoir” as used herein refers to a chamber element within a device which is designed to comprise liquids or semi-liquids such as gels or matrix material. The reservoir may have any suitable size or shape. It serves to delimit liquid and/or matrix filled spaces from each other, wherein a reservoir according to the invention is typically in communication with another reservoir within the device. Such communication may be implemented by openings, inlets, outlets, tubings or functional units etc. They may be present at different locations within the reservoirs and be of different sizes and/or different functionality. The specimen carrier may comprise one or more of the reservoirs, e.g. 1-10, 1-20, 1-100, 1-500 or any numbers any between these integers. The specimen carrier may, for example, comprise a number of reservoirs which correspond to the number of wells in typical microwell plates such as 12, 24, 48, 96, 192, 384 etc.

The term “segmented inner wall” as used herein refers to a wall structure which comprises sub-structures that form segments. Such segments serve as meniscus barriers and are designed to laterally stabilize enclosed gels or gel matrix substances within the reservoir. Theses structures further prevent the meniscus formation of liquids or gel matrix materials filled into said reservoir.

In preferred embodiments the specimen carrier is hence a multi-well carrier. The specimen carrier may accordingly comprise any suitable number of wells, wherein the term “well” as used herein refers to a reservoir as mentioned herein, preferably to a unit of an inner and outer reservoir as defined herein. For example, the multiwell carrier may preferably comprise 12, 24, 48 or 96 wells. Also envisaged are a higher number of wells such as 384, 512, 768, 1024 or 1536 wells.

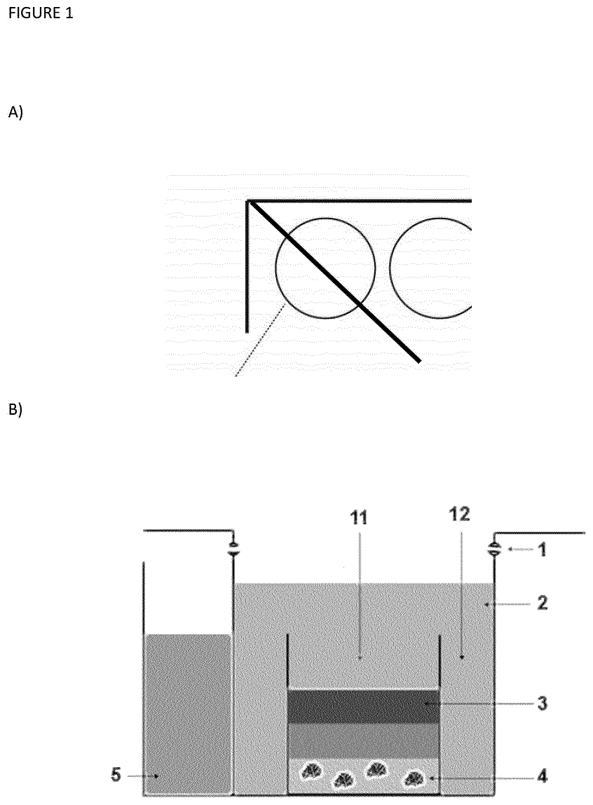

According to specific embodiments of the present invention the reservoir comprises an inner reservoir (11) and an outer reservoir (12). The term “inner reservoir” as used herein, refers to a reservoir which is at least partially embedded or enclosed by a surrounding structure, which is term “outer reservoir”. An example of a specimen carrier comprising an inner and outer reservoir is provided in FIG. 1B, where reference sign 11 depicts an inner reservoir, whereas reference sign 12 depicts an outer reservoir. The inner reservoir is typically designed the comprise a gel matrix, optionally also liquid medium or cell culture medium. The inner reservoir is accordingly also designated herein as matrix reservoir The outer reservoir is typically designed to comprise a liquid medium. The outer reservoir is accordingly also designated herein as medium reservoir. An inner wall structure as defined herein is preferably present at the inner wall of the inner reservoir. Both reservoir types are in liquid communication, preferably via the top opening of the inner reservoir, which allows for a liquid exchange between the inner and outer reservoir.

According to certain embodiments of the invention the inner reservoir has a symmetric or asymmetric position with respect to the outer reservoir in plan view. A symmetric position of the inner reservoir with respect to the outer reservoir may be implemented by a centered position of the inner reservoir within the outer reservoir, preferably with equal distances between the walls of the inner reservoir and the walls of the outer reservoir. An asymmetric position of the inner reservoir with respect to the outer reservoir may be implemented by an eccentric position with respect to center of the outer reservoir. In an eccentric position, the outer reservoir may benefit from more space for a more effective exchange of liquid by improved accessibility with a pipette.

The specimen carrier accordingly implements an overall structure wherein the wall of the inner reservoir is shorter than the wall of the outer reservoir, thus allowing an enclosing of the entire inter reservoir by a medium, which is present in the outer reservoir. Media changes can be performed, for example, by tilting the plate with accessing primarily the outer medium reservoir with the pipette.

In preferred embodiments the cell culture medium present in the outer reservoir overlays compounds or elements present in the inner reservoir. It is particularly preferred that the cell culture medium as present in outer reservoir overlays a matrix layer present in the inner reservoir. This allows for a communication with the matrix reservoir.

According to certain embodiments of the present invention the inner reservoir may comprise, when used, a layered succession of material, wherein at the bottom layer a gel matrix is located, which is overlaid by a medium or cell medium layer or additional gel matrix layers.

It is particularly preferred that the inner reservoir comprises an inner wall structure which prevents the meniscus formation of liquids or gel matrix and stabilizes the gel mechanically as described herein below.

According to further embodiments the one or more reservoirs may comprise a ventilation opening. The term “ventilation opening” as used herein refers to a hole, outlet or valve element, which allows for exchange of gas, e.g., of air or vapor between one or more reservoirs, or between the reservoirs and the outside.

The ventilation opening is, in certain embodiments, designed to connect to an adjacent external reservoir, e.g. a humidity reservoir, and accordingly allows for the increase of local humidity.

In further specific embodiments, the ventilation opening may alternatively or additionally be designed to connect to an adjacent external reservoir and to allow for the additional exchange of liquid, e.g. medium between the reservoirs.

In certain further embodiments, the ventilation opening may be non-controllable or static entity which is provided in an open form and allows for permanent gas exchange. Alternatively, the ventilation opening may be controllable, e.g. by electronic or computer modules, preferably via remote control so that the gas exchange can be deliberately be managed and controlled. The ventilation opening may further be connected to gas, vapor or air component detectors which may be present throughout the reservoirs and which allow to determine the amount of target gases which may lead to an opening or closing of the ventilation openings.

In a further preferred embodiment, the specimen carrier comprises, besides an inner (e.g. matrix reservoir) and an outer reservoir (e.g. medium reservoir), an additional liquid reservoir, which is preferably an adjacent liquid reservoir. This reservoir may fulfil the function of a humidity, liquid or multipurpose reservoir). In certain embodiments, this additional reservoir may be placed outside of a unit comprising an inner and outer reservoir. It may be in aerial communication with the inner reservoir and the outer reservoir, e.g. with the above mentioned ventilation opening. Alternatively or additionally, it may in liquid communication with the inner reservoir and the outer reservoir, e.g. as inlets/outlets, or, in certain embodiments, via the ventilation openings.

The reservoir may be designed to receive liquid, e.g., distilled water or cell culture medium, which may be used to refill the unit comprising an inner and outer reservoir, or which functions as spillover reservoir in case the liquid in the inner and outer reservoir climbs beyond an upper limit, e.g. defined by the position of an outlet or opening. The additional reservoir may further be used to provide supplements, drugs, medicaments, protein factors etc., which are required by cells, which may grow within the inner reservoir. These factors may, for example, be introduced into the unit of outer and inner reservoir via an opening or inlet between the additional reservoir and the outer reservoir.

In further embodiments, the reservoir may have any suitable form or shape. It may, for example, be in a circular, rectangular, trapezoid, ellipsoid, or roundly shaped transversal form. In plan view, the inner and outer reservoir may have circular, rectangular, trapezoid, ellipsoid or roundly shaped form. The form or shape may be adapted to or derived from the overall form or shape of the specimen carrier or may be independent therefrom. In further embodiments, a reservoir in plan view a circular transversal form may have a cylindric, trapezoid, pyramidal or conical shape in the transversal. In yet another group of embodiments, a reservoir of a rectangular transversal form may have a cuboidal or pyramidal shape, e.g. an inverse pyramidal shape.

The reservoirs comprise an inner wall structure, which is designed to prevent meniscus formation of liquids or gel matrix material. A “meniscus” as used herein refer to a curve in the upper surface of a liquid or semi-liquid material such as a gel matrix close to the surface of the chamber element, which is produced by surface tension. The meniscus is typically a concave meniscus, which occurs when the attraction (adhesion) between the particles of the liquid or semi-liquid material and the chamber element, e.g. a reservoir, is more than half the attraction (cohesion) of the particles of the liquid to each other, which causes the liquid or semi-liquid material to climb the walls or in the case of a convex surface less than half or descends the wall (e.g. via repulsion) of the chamber element, e.g. reservoir. Without wishing to be bound to theory, it is assumed that the generation of menisci is further governed by physical parameters of a system of a solid (e.g. the wall structure of a reservoir), a liquid and a vapor at a given temperature and pressure. The liquid is typically assumed to build a “contact angle” between the solid and the liquid surface. This angle is measured between the surface tangent on the liquid-vapor interface and the tangent on the solid-liquid interface at their intersection. The contact angle may, for example, be calculated with the Young equation or the Young-Dupre equation. In the context of vertical solid surfaces such as wall structures in a reservoir according to the present invention the interface between the liquid and the vapor (e.g. air) forms a curved shape, wherein said shape is dependent on the magnitude of the contact angle. This shape corresponds to a meniscus as described above.

Since meniscus formation is considered disadvantageous for cell culturing and within the context of provision and handling of extracellular matrices, it is one objective of the present invention to prevent meniscus formation of liquid or gel matrix material in a reservoir according to the present invention. The term “prevention of meniscus formation” as used herein means that the curvature of the meniscus is reduced, preferably completely eliminated, i.e., it is converted to a flat or almost flat surface. The curvature may, for example, be reduced to a value of 1/r of 0.001/m, e.g., via a modification of the adhesion effects of liquid or semi-liquid particles. It is particularly preferred to obtain a contact angle of 90° which is characteristic of a flat liquid surface or eliminated meniscus. This reduction may be achieved by chemical adhesion parameters, e.g. the coating of walls and/or by geometrical or mechanical structures within the solid surface, e.g. a wall within the reservoir as defined herein. One of these options or one combination of these options fulfils the function of a “meniscus barrier”, i.e., the generation of menisci within the specimen carrier is reduced or eliminated. According to the present invention the increased segmentation of the inner wall structure allows a seamless filling with liquid or gel and prevents the rise of a meniscus through the angle of the barrier.

According to the present invention the reservoirs comprise meniscus barriers in the form of “inner wall structures”. These structures are typically present at the vertical dimension, e.g., as vertical walls of the one or more reservoirs. They may comprise different forms and functions, which modify the contact angle as defined herein in a mechanical and/or physicochemical way. Preferably, the inner wall structures may be a vertical barrier, a circular barrier on a vertical wall, a linear barrier on a vertical wall.

In preferred embodiments, more than one meniscus barrier may be present at an inner wall. Accordingly, a group of 2, 3, 4, 5, 6, 7, 8, 9, 10, 15, 20, 25, 30, 35, 40, 50, 100 or more meniscus barriers, or any value in between the mentioned values of meniscus barriers, may be present at an inner wall. In further embodiments, one of these meniscus barriers may be placed in defined distance from a further of these barriers. This distance may be, for example, a vertical distance of 40 μm-2 mm, e.g. a vertical distance of 40 μm, 50 μm, 60 μm, 70 μm, 80 μm, 90 μm, 100 μm, 150 μm, 200 μm, 250 μm, 300 μm, 350 μm, 400 μm, 450 μm, 500 μm, 600 μm, 700 μm, 800 μm, 900 μm, 1 mm, 1.5 mm, 1.75 mm or 2 mm.

In case, more than two meniscus barriers are present at the inner wall of a reservoir, the distance between these barriers may be the same or may be different, e.g. any distance as described above.

According to specific embodiments, the meniscus barrier is a mechanical meniscus barrier. The term “mechanical meniscus barrier” as used herein refers to a 3-dimensional structure, which is present at an inner wall of a reservoir according to the present invention, which provides a mechanical effect on the contact angle between liquid/semi-liquid and solid structure and which, in consequence, reduces the curvature of the meniscus or eliminates the meniscus.

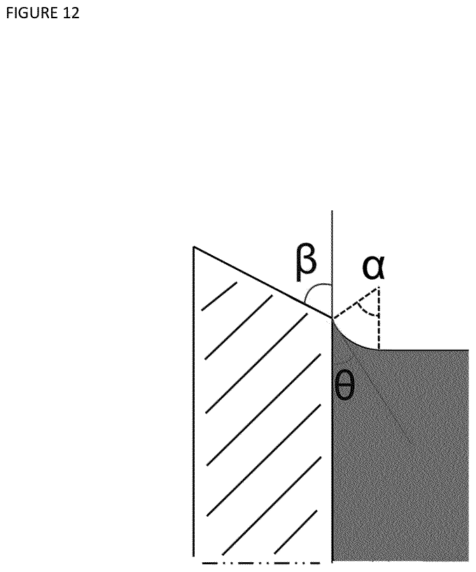

Without wishing to be bound to theory, it is assumed necessary that the mechanical structure that prevents propagation of the meniscus shows a geometric structure that fulfills the following criterion:

$$Δ P = \frac{2 {γ}_{la}}{ω} ( \frac{cos θ - \frac{α}{sin α}}{\frac{1}{sin α} ( \frac{α}{sin α} - cos α )} ) \qquad ( Formula 1 )$$

wherein alpha is the meniscus arc angle, beta the angle of the said mechanical structure in relation to the surface of the reservoir wall and theta the sharpest angle of the meniscus. Further details would be known to the skilled person or can be derived from suitable literature references such as Papadimitriou et al., 2018, Analytica Chimica Acta, 1000, 232-238. An illustration of the geometry of meniscus formation may be derived from FIG. 12. Examples of such a 3-dimensional structure include a wall indentation, a wall protuberance, a recess in the wall or a shoulder, which is present in the inner wall.

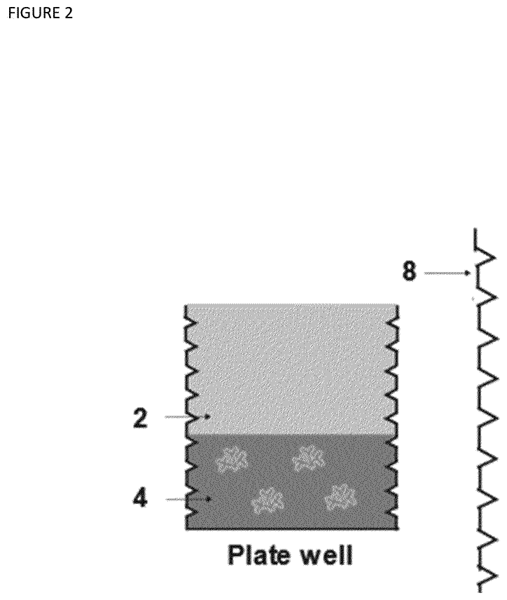

The indentations may have any suitable form. Examples of indentations according to the present invention are depicted in FIG. 2, which shows a variation of an indentation with reference sign 8.

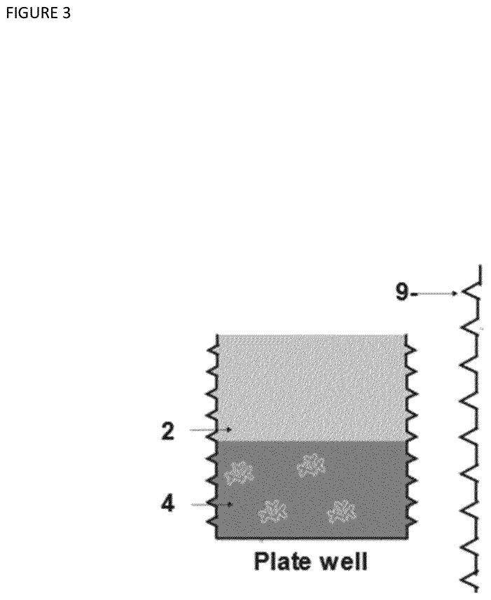

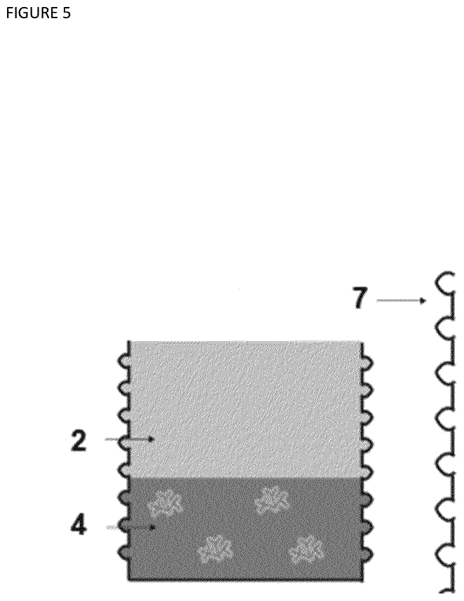

The protuberance may have any suitable form, preferably, it may be a pointed protuberance or a curved protuberance. Examples of protuberances according to the present invention are depicted in FIG. 3, which shows a variation of a pointed protuberance with reference sign 9, as well as FIG. 5, which shows a variation of a curved protuberance with reference sign 7.

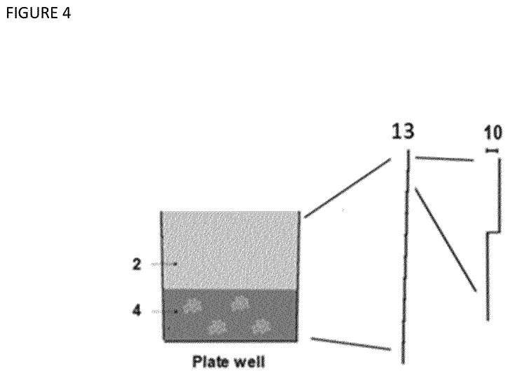

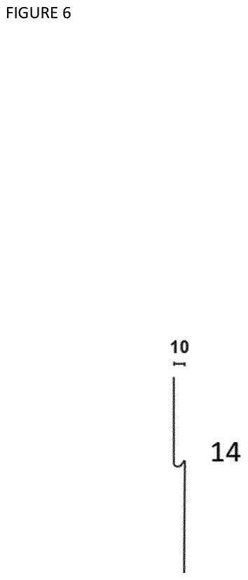

The recess may have any suitable form. An example of a recess according to the present invention is depicted in FIG. 4, which shows a variation of a recess with reference sign 13. Further examples can be derived from FIG. 6 which shows a further variation of a recess with reference sign 14.

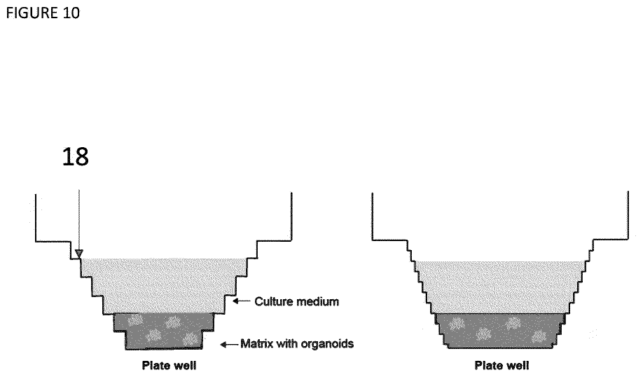

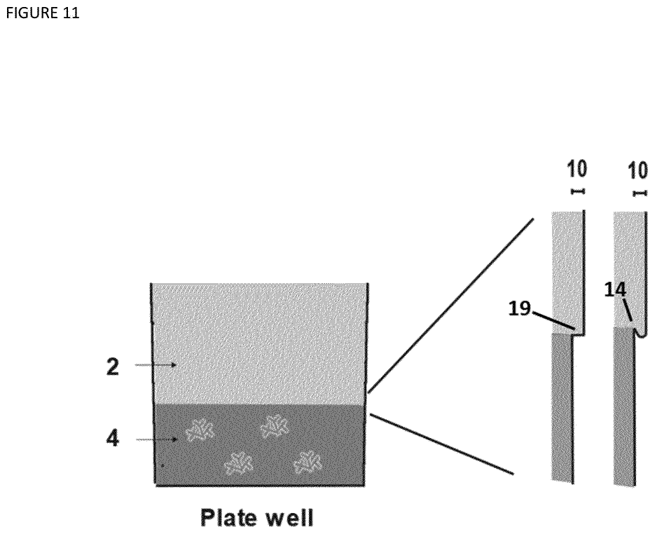

The shoulder may have any suitable form. An example of a shoulder according to the present invention is depicted in FIG. 10, which shows a variation of a shoulder with reference sign 18. Further examples can be derived from FIG. 11 which shows a further variation of a shoulder with reference sign 19 and 14. In preferred embodiments, the angle between the vertical and non-vertical part of the shoulder an angle of about 90°. In further embodiments, the angel may also be less than 90°, e.g., 85°, 75°, 70° or any other suitable angle, or any value in between the mentioned angles. In yet another embodiment, the angel may also be more than 90°, e.g., 95°, 105°, 110° or any other suitable angle, or any value in between the mentioned angles.

In particularly preferred embodiments, the shoulder type or recess type of the meniscus barrier may comprise a vertical tip, e.g., as depicted in FIG. 6. The tip may be vertically sharp, e.g., comprising an angle of 1-15°. In further embodiments, the tip may alternatively or additionally have a base width of about 1 μm to 100 μm, e.g., 10 μm, 20 μm, 30 μm, 40 μm, 50 μm, 60 μm, 70 μm, 80 μm, 90 μm or 100 μm or any value in between the mentioned values.

According to another group of specific embodiments, the meniscus barrier is a chemical meniscus barrier. The term “chemical meniscus barrier” as used herein refers to a barrier, which is present at an inner wall of a reservoir according to the present invention, which implements a chemical interaction between the liquid or semi-liquid and the solid structure of the reservoir, thereby modifying the contact angle between liquid/semi-liquid and solid structure which, in consequence, reduces the curvature of the meniscus or eliminates the meniscus. Examples of such chemical meniscus barriers include alternating hydrophobic and hydrophilic surfaces of the wall, e.g., vertically and/or horizontally oriented stretches or zones, wherein hydrophobic molecules are placed, followed by stretches or zones, where hydrophilic molecules are placed. Theses zones may have any suitable form or size. Examples of hydrophobic molecules include manganese oxide polystyrene (MnO₂/PS) nano-composite, zinc oxide polystyrene (ZnO/PS) nano-composite, precipitated calcium carbonate, carbon nano-tube structures, silica nano-coating, fluorinated silanes and fluoropolymer coatings. Further, examples of hydrophilic molecules include uncoated polypropylene, polystyrene, polycarbonate, glass, quartz or coated with polyvinylpyrolidone, polyurethanes, polyacrylic acid, polyethylene oxide and polysaccharides.

In specific embodiments of the present invention the chemical meniscus barriers comprise a coating. The term “coating” as used herein refers to chemical substances, which can be applied to the solid substrate, i.e., the wall of a reservoir as described herein. Examples of such chemical substances include fluoropolymer agents, siliconizing agents, waxes such as paraffin waxes, polyolefin waxes, vegetable waxes, saturated fatty acids, oleic acids, alpha-linoleic acid, linoleic acid, arachidonic acid, eicosapentaenoic acid, docosahexaenoic acid, erucic acid, fatty alcohols, hexafluoropropylene (HFP), vinylidene fluoride (VDF), tetrafluoroethylene (TFE), vinylidene fluoride (VDF), hexafluoropropylene (HFP), perfluoromethylvinylether (PMVE), silicon, fluorosilicon, neoprene, urethane, EPDM, siloxane, methylsiloxane or methylvinyl siloxane and others. The coating of the reservoir structures may be performed according to methods known to the skilled person. It is preferred that the coated material does not emit or release any of the coating substances into the liquid or semi-liquid.

The coating may be present at specific zones of the inner wall.

The alteration of zones with chemical barriers may be, in one set of embodiments, a linear alteration. For example, in a vertical and/or horizontal dimension a hydrophilic zone follows a hydrophobic zone which follows a further hydrophilic zone etc., or a coated zone follows a non-coated zone, which follows a further coated zone etc. Alternatively, the alteration may be non-linear, e.g., two separate hydrophilic zones or coated zones are followed by one hydrophobic zone or non-coated zone or vice versa. Further, circular arrangement of zones or asymmetric arrangements are envisaged herein. It is preferred that coated zones are organized in a repeating form at the inner wall, e.g., a coated zone is followed by non-coated zone in a vertical direction.

According to further embodiments of the invention mechanical and chemical meniscus barriers may be present within one reservoir, e.g., the inner reservoir. These barrier types may be combined in any suitable manner, e.g., a mechanical barrier is followed by a chemical barrier, which is followed by a mechanical barrier etc. Alternatively, the mechanical barriers may be provided a the bottom of the inner wall and the chemical barriers may be provided at the top of the inner wall, or vice versa.

According to further embodiments of the invention the meniscus barrier may have any suitable horizontal dimension. The term “horizontal dimension” as used herein means that the structure which is present at the inner wall of a reservoir according to the present invention covers a certain area of the inner wall, which comprises a horizontal component. The horizontal dimension may be adjusted to the form, shape, size of the specimen carrier, in particular of the reservoir as described above. In preferred embodiments the horizontal dimension of the meniscus barrier may be 5 μm to 2 mm, e.g. 5 μm, 10 μm, 15 μm, 20 μm, 25 μm, 30 μm, 50 μm, 60 μm, 70 μm, 80 μm, 90 μm, 100 μm, 110 μm, 120 μm, 130 μm, 150 μm, 200 μm, 300 μm, 400 μm, 500 μm, 600 μm, 700 μm, 800 μm, 900 μm, 1000 μm, 1100 μm, 1200 μm, 1300 μm, 1400 μm, 1500 μm, 1600 μm, 1700 μm, 1800 μm, 1900 μm, 2000 μm or any value in between the mentioned values. In a particularly preferred embodiment, the horizontal dimension is between 5 μm to 500 μm.

In yet another set of embodiments of the invention the meniscus barrier may have any suitable vertical dimension. The term “vertical dimension” as used herein means that the structure which is present at the inner wall of a reservoir according to the present invention covers a certain area of the inner wall, which comprises a vertical component. The vertical dimension may be adjusted to the form, shape, size of the specimen carrier, in particular of the reservoir as described above. In preferred embodiments the vertical dimension of the meniscus barrier may be 30 μm to 2 mm, e.g. 30 μm, 50 μm, 60 μm, 70 μm, 80 μm, 90 μm, 100 μm, 110 μm, 120 μm, 130 μm, 150 μm, 200 μm, 300 μm, 400 μm, 500 μm, 600 μm, 700 μm, 800 μm, 900 μm, 1000 μm, 1100 μm, 1200 μm, 1300 μm, 1400 μm, 1500 μm, 1600 μm, 1700 μm, 1800 μm, 1900 μm, 2000 μm or any value in between the mentioned values. In a particularly preferred embodiment, the vertical dimension is between 30 μm to 500 μm.

According to embodiments of the invention the specimen carrier comprises a multitude of reservoirs, wherein this multitude of reservoirs is connected, in one embodiment, by an aerial distribution system based on the distribution of humidity and/or, in a further embodiment, b a liquid distribution system. The invention also envisages the presence of both distribution systems in one specimen carrier.

The term “aerial distribution system” as used herein refers to a chamber entity, which encloses or partially encloses reservoirs as defined herein It may, in certain embodiments, be designed to work as humidity reservoir. It is accordingly in aerial communication with one or more reservoirs of the carrier, preferably though ventilation openings (1). The system may, for example, be filled with liquid such as distilled water, which can evaporate and maintain or increase the humidity in the reservoir system.

The term “liquid distribution system” as used herein refers to a chamber entity, which encloses or partially encloses reservoirs as defined herein. The system may, for example, be in liquid communication with one ore more reservoirs of the carrier, preferably e.g. via inlets or outlets, in certain specific embodiments through ventilation openings (1). In this embodiment, the liquid distribution system may comprise, for example, culture medium, optionally also comprising supplements, drugs, growth factors etc. The system may, in embodiments of the invention, surround the reservoirs and allows for the introduction of the liquid into one, more or all of the units of inner and outer reservoirs. The connection between the liquid distribution system and the unit of inner and outer reservoir may be implemented via inlets or outlets. The present invention further envisages that the liquid distribution system comprises one or a multitude of additional liquid reservoirs (5) as defined herein.

In a further preferred embodiment the carrier comprises an access port. The “access port” as used herein refer to an inlet, outlet or opening which is designed to be connected to a tubing, e.g., being part of a clinical diagnosis device, or which designed to be accessible via a syringe or the like so that a liquid may be injected into the device that is distributed in the humidity reservoir. The access port may further be connected to a pumping and/or microfluidic system allowing for the introduction and, in certain embodiments, for the removal of elements into and/or out of the carrier. In certain embodiments the access port is designed for a syringe based injection of liquids, e.g. comprising supplements, nutrients, drugs, protein factors or the like.

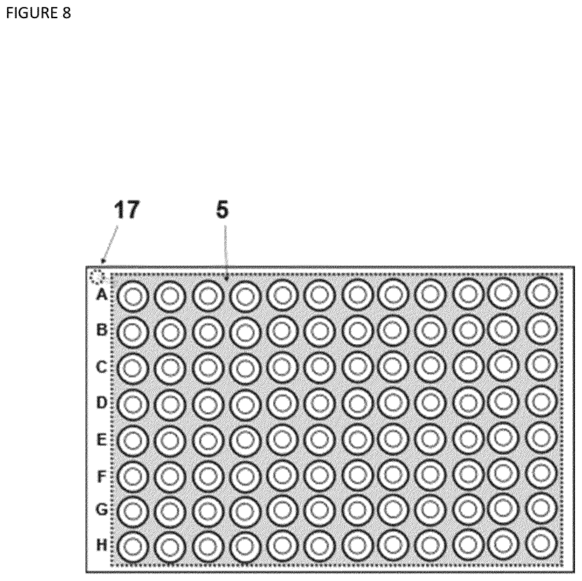

In certain embodiments the access port for liquid injection is located at any suitable location of the carrier, preferably at an edge or corner of the carrier. An example of a preferred position of the access port can be seen in FIG. 8, wherein the access port is designated with reference number 17.

In a further set of embodiments, the bottom of a reservoir may be transparent. It is preferred that the transparent bottom is capable of allowing for the performance of microscopy analysis. In further preferred embodiments the inner or outer reservoir may have a transparent bottom. In yet another group of embodiments, both, the inner and outer reservoir may have a transparent bottom. The transparency of the bottom may be achieved by the use of transparent plastic material or glass material. Further, the specimen carrier may comprise lighting units and/or tubings or chambers, where lighting units may be positioned. It is also envisaged that camera or microscope units be placed in or on the carrier. Also contemplated is that adaptors for microscopic management be provided in or on the specimen carrier. Further, the carrier may comprise barcode sections, QR-code sections, RFID chips or other components which allow for an automatized operation and storage or retrieval of the specimen carrier.

In a further aspect the present invention relates to the use of the specimen carrier as defined herein for culturing cells. The specimen carrier may, for example, be used for the growth and culturing or eukaryotic cells, prokaryotic cells, preferably of human or mammal cells. The carrier may accordingly be placed in a suitable environment wherein adequate temperature, gas and/or shaking conditions are provided. Preferred conditions include, for example, 37° C. and 100% humidity. Further information would be known to the skilled person or can be derived from suitable textbooks such as Alberts et al., 2002, Molecular Biology of the cell, 4^{th} ed, Garland Science.

In yet another aspect the current invention relates to a method for the culturing of cells, comprising: (i) providing a specimen carrier as defined herein (ii) filling the reservoirs with matrix gel and optionally culture liquid; and (iii) culturing cells within the specimen carrier.

The term “matrix gel” as used herein relates to extracellular matrix material, which allows cell culture cells to settle and thrive within a reservoir. Preferred matrix gels include, for example, gels derived from Engelbreth-Holm-Swarm (EHS) mouse sarcoma cells (further details can be derived from https://www.ncbi.nlm.nih.gov/pmc/articles/PMC9733768/; last visited Dec. 18, 2023) such as Matrigel (Corning) or Basement membrane extract (BME) (Cultrex) or synthetic matrix gels such as hyaluronic acid based Hystem or Trugel3D (Sigmaaldrich).

In preferred embodiments step (iii) may be performed for any suitable period of time. This period of time may be adapted to the type of cell to be cultured. It may, in certain embodiments, comprise 1, 2, 3 days up to 4, 8, or 12 weeks or any period in between the mentioned periods.

The following figures are provided for illustrative purposes. It is thus understood that the figures are not to be construed as limiting. The skilled person in the art will clearly be able to envisage further modifications of the principles laid out herein.

1. A specimen carrier, comprising:
one or more reservoirs, wherein said reservoirs comprise a segmented inner wall structure (meniscus barrier) which laterally stabilizes an enclosed gel and prevents meniscus formation of a liquid or gel matrix material filled into said reservoir.

2. The specimen carrier of claim 1, wherein the inner wall structure comprises at least two, preferably a multitude of, more preferably 10 to 20, vertically arranged meniscus barrier(s), wherein said barrier is designed to prevent near seamless formation of a meniscus of liquids or gel matrix material filled into said reservoir and to stabilize the gel by increasing the contact surface and to strengthen lateral adhesion.

3. The specimen carrier of claim 2, wherein said meniscus barrier is a vertical, circular, linear, mechanical and/or a chemical meniscus barrier, or a combination thereof, preferably with a vertical distance of 40 μm-2 mm to a further barrier.

4. The specimen carrier of claim 3, wherein said mechanical meniscus barrier is an inner wall indentation, an inner wall protuberance, preferably a pointed or curved protuberance, a recess or a shoulder, preferably a shoulder comprising an angle of about 90°, or less than 90°, or more than 90°.

5. The specimen carrier of claim 4, wherein the shoulder meniscus barrier has a horizontal dimension of 5 μm to 2 mm, preferably 5 μm to 500 μm, and a vertical dimension of 5 μm-2 mm.

6. The specimen carrier of claim 4, wherein the shoulder meniscus barrier comprises a vertical tip, preferably of an angle of 1-15° and/or a base width of 1 μm to 100 μm.

7. The specimen carrier of claim 3, wherein said chemical barrier is an alternating hydrophobic and hydrophilic surface of the wall.

8. The specimen carrier of claim 3, wherein a mechanical and a chemical barrier is combined.

9. The specimen carrier of claim 1, wherein the inner wall of the reservoir comprises a coating, preferably a coating which is present at specific zones of the inner wall, more preferably at repeating zones followed by non-coated zones in vertical direction.

10. The specimen carrier of claim 1, wherein the reservoir comprises an inner reservoir (11) and an outer reservoir (12), wherein the inner reservoir comprises the inner wall structure that laterally stabilizes the gel which prevents the meniscus formation of liquids or gel matrix.

11. The specimen carrier of claim 10, wherein the inner reservoir (11) is designed to receive a gel matrix and optionally cell culture medium (matrix reservoir) and wherein the outer reservoir (12) is designed to receive cell culture medium (medium reservoir).

12. The specimen carrier of claim 11, wherein the cell culture medium present in the outer reservoir overlays a matrix layer present in the inner reservoir.

13. The specimen carrier of claim 1, wherein the reservoir, preferably the medium reservoir (12), comprises a ventilation opening (1).

14. The specimen carrier of claim 1, wherein the inner reservoir (11) and the outer reservoir (12) are in liquid or aerial communication with an additional liquid reservoir (5), preferably an adjacent liquid reservoir.

15. The specimen carrier of claim 1, wherein the medium reservoir (12) is in liquid communication with the matrix reservoir (11).

16. The specimen carrier of claim 1, wherein the carrier comprises a liquid distribution system adjacent to the reservoir(s) which is in liquid or aerial communication with all reservoirs of the carrier, preferably though ventilation openings (1).

17. The specimen carrier of claim 1, wherein the carrier is a multi-well carrier, preferably a 12, 24, 48 or 96 well carrier.

18. The specimen carrier of claim 1, wherein the reservoir has a circular, rectangular, ellipsoid, trapezoid or roundly shaped transversal form.

19. The specimen carrier of claim 18, wherein the reservoir of a circular transversal form has a cylindric or conical shape, or wherein the rectangular transversal form has a cuboidal or pyramidal shape.

20. The specimen carrier of claim 1, wherein the carrier comprises an access port for liquid injection, preferably at an edge or corner of the carrier.

21. The specimen carrier of claim 10, wherein the inner reservoir has a symmetric or asymmetric position with respect to the outer reservoir in plan view, preferably having a circular, rectangular, trapezoid, ellipsoid, or roundly shaped form in plan view.

22. The specimen carrier of claim 21, wherein said asymmetric position is an eccentric position with respect to center of the outer reservoir in plan view.

23. The specimen carrier of claim 1, wherein the bottom of the reservoir, preferably of the inner (11) and/or outer reservoir (12), is transparent, preferably designed for microscopy analysis.

24. (canceled)

25. (canceled)

26. Method for the culturing of cells, comprising: (i) providing a specimen carrier as defined in claim 1 (ii) filling the reservoirs with matrix gel and optionally culture liquid; (iii) culturing cells within the specimen carrier, preferably for a time period of 1, 2, 3 days up to 4, 8, or 12 weeks.

27. Method for the microscopic analysis of living cells or tissues under culture conditions, comprising: (i) culturing cells within the specimen carrier providing a specimen carrier as defined in claim 1 (ii) performing microscopic analysis of cells embedded in a gel matrix in the specimen carrier.
